(19) United States
(12) Patent Application Publication
(10) Pub. No.: US 2026/0335484 A1
(43) Pub. Date: Oct. 29, 2026

(54) SMART PROGRAMMABLE ULTRAVIOLET GERMICIDAL IRRADIATION SYSTEM AND METHOD

(71) Applicant: Arizona Board of Regents on Behalf of the University of Arizona, Tucson, AZ (US)

(72) Inventor: Stanley PAU, Tucson, AZ (US)

(21) Appl. No.: 19/109,113

(22) PCT Filed: Sep. 8, 2023
(86) PCT No.: PCT/US2023/073743

§ 371 Date: Mar. 5, 2025

Related U.S. Application Data
(60) Provisional application No. 63/374,975, filed on Sep. 8, 2022.

(51) Int. Cl. A61L 2/24; A61L 2/104; A61L 103/95

(52) U.S. Cl. CPC A61L 2/24; A61L 2/104; A61L 2103/95; A61L 2202/11; A61L 2202/14; A61L 2202/16

(57) ABSTRACT

Methods, devices and systems for precise delivery of an exact dose of ultraviolet (UV) radiation for disinfecting to an area are disclosed. One example method for disinfecting one or more objects includes obtaining an image of an enclosure and information indicative of a material property of the objects and a type of contaminant to be disinfected. The disinfection method further includes determining an irradiance and a shape of UV illumination, and operating a UV radiation system to illuminate each of the objects according to the determined irradiance and shape of the UV illumination for a predetermined period of time. The enclosure is monitored by obtaining additional images, and analyzing them to determine whether a change in the enclosure is detected. The UV radiation can be topped or modified upon expiration of the predetermined period of time, or based on the analysis of the images.

### CROSS-REFERENCE TO RELATED APPLICATION(S)

This application claims priority to the provisional application with Ser. No. 63/374,975 titled “SMART PROGRAMMABLE ULTRAVIOLET GERMICIDAL IRRADIATION SYSTEM AND METHOD,” filed Sep. 8, 2022. The entire contents of the above noted provisional application are incorporated by reference as part of the disclosure of this document.

### TECHNICAL FIELD

The disclosed technology relates to disinfection methods and systems that utilize ultraviolet radiation.

### BACKGROUND

Removal or neutralization of pathogens from air and surface inside a shopping mall, airport, hospital, classroom, restaurant, nursing home and store can reduce odor, allergy reaction and transmission of infectious diseases. Disinfection can be carried out in a variety of ways, for example by using heat sterilization, chemicals, air filter, alternating electrostatic fields, ozone, or ultraviolet (UV) radiation. Among the many techniques, ultraviolet germicidal irradiation (UVGI) which utilizes ultraviolet C (UVC) light (200-280 nm) with optimal wavelength at around 265 nm is the most common. The emergence of SARS-CoV-2 in 2019 created a pandemic that led to a large demand for deployable solutions to reduce and control viral transmission.

### SUMMARY

The disclosed embodiments relate to methods, devices and systems that enable precise delivery of an exact dose of UV radiation to an area of interest. The disclosed smart programmable sanitizers can be operated autonomously and remotely with minimal user intervention.

One example method for disinfecting one or more objects using UV radiation includes obtaining an image of an enclosure and information indicative of a material property of the one or more objects to be disinfected within the enclosure and a type of contaminant to be disinfected, and determining an irradiance and a shape of UV illumination for each of the one or more objects to be disinfected based on the obtained image, the information indicative of a material property of the one or more objects and the type of contaminant. The method further includes operating a UV radiation system to illuminate each of the one or more objects with UV radiation according to the determined irradiance and shape of the UV illumination for a predetermined period of time, monitoring the enclosure by obtaining additional images of the enclosure, analyzing the additional images to determine whether a change is detected in the enclosure, and unless analysis of the additional images indicates that illumination of the one or more objects should be stopped or modified prior to expiration of the predetermined period of time, stopping or modifying the UV radiation upon expiration of the predetermined period of time.

### BRIEF DESCRIPTION OF THE DRAWINGS

FIGS. 1A to 1C illustrate different examples of an ultraviolet germicidal irradiation (UVGI) system.

FIG. 2 illustrates a smart programmable ultraviolet germicidal irradiation system in accordance with an example embodiment.

FIG. 3 illustrates a smart programmable ultraviolet germicidal irradiation system in accordance with another example embodiment.

FIGS. 4A to 4D illustrate different ultraviolet (UV) coverage arrangements for the UV sanitizer in accordance with example embodiments.

FIG. 5 is an example diagram illustrating the surface of a target with a normal direction defined by a vector.

FIG. 6 illustrates a set of operations that can be carried out to disinfect an area with UV radiation in accordance with an example embodiment.

FIG. 7 illustrates a steering wheel that is disinfected in accordance with an example embodiment.

FIGS. 8A and 8B illustrate a cell phone and an augmented reality/virtual reality (AR/VR) goggle device which can be utilized to control a UV disinfection system in accordance with example embodiments.

FIG. 9 illustrates user interfaces for controlling a UV disinfection system in accordance with example embodiments.

FIG. 10 illustrates marking of objects inside the virtual environment for UV disinfection according to an example embodiment.

FIG. 11 illustrates a set of operations that can be carried out to disinfect one or more objects using UV radiation in accordance with an example embodiment.

### DETAILED DESCRIPTION

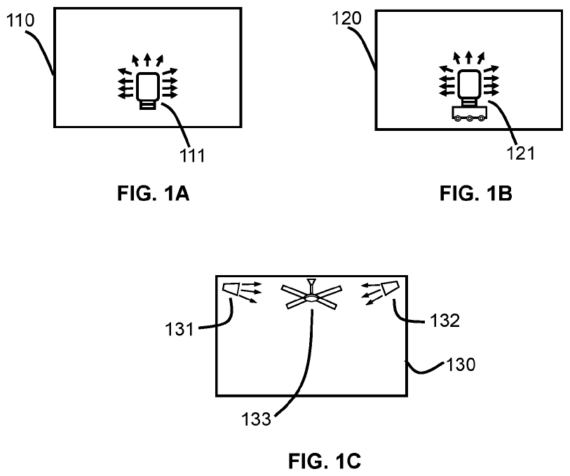

FIGS. 1A to 1C show different types of UVGI system inside a room. The simplest configuration is shown in FIG. 1A where a single UVC light source, 111, is utilized to illuminate an entire room 110. The light source 111 can be fixed or portable. FIG. 1B shows another configuration where a light source is mounted on an autonomous vehicle, 121, which can turn on and off and navigate inside the room 120. The autonomous vehicle is shown to be moving on the floor, but it can also be positioned on rail on the ceiling. FIG. 1C shows multiple light sources, 131 and 132, mounted on the ceiling of a room 130. The light sources are positioned to illuminate only the top part of the room, e.g., the ceiling. A fan 133 is utilized to create air flow from the bottom of the room to the top of the room, where the irradiation occurs. This upper-air UVC system performs disinfection above the heads of the occupants and tends to have a low UVC flux and flow rate, resulting in ventilation of 2 to 6 air change per hour (ach).

Existing UVGI systems tend to be imprecise, static and non-programmable and illuminate different areas indiscriminately and uniformly. While such systems tend to be low cost and can be hand operated by a user to apply to different surfaces, there is a need to develop a smart programmable sanitizer that can be operated autonomously and remotely with minimal user intervention. A programmable sanitizer can precisely deliver an exact dose of UV radiation to an area and can be safer than existing UVGI system.

Currently the most efficient source of UVC light in UVGI systems is a low-pressure mercury discharge lamp, with about 85% of the energy at 253.7 nm, which is close to 265 nm -the optimal wavelength for inactivation of microorganisms. It has been shown that a dose of about 25000 μW-s/cm² per log of kill is needed to inactivate bacillus subtilis endospores at 254 nm. For 6 log kill, a dose of 150000 μW-s/cm² is more than enough to kill other viruses and bacteria, such as influenza A, mycobacterium tuberculosis and escherichia coli. It has also been shown that a dose of 40 mW-s/cm² is sufficient to achieve a 6 log kill of SARS-CoC-2. State-of-the-art UVC LEDs currently have about 6% efficiency and 10000 hours of lifetime, compared to mercury lamps which have about 30-40% efficiency and 9000 hours of lifetime. However, UVC LED is catching up fast and is anticipated to have a similar efficiency as the mercury lamp within 10 years. UVC LEDs also have many additional advantages over lamps, including having a long lifetime, being environmentally friendly in disposal, being compact and having a flexible geometry. Non-imaging optical designs that are otherwise not possible when using conventional lamp sources can be implemented using UVC LEDs by fully taking advantage of the light distribution and shape of the UVC LED.

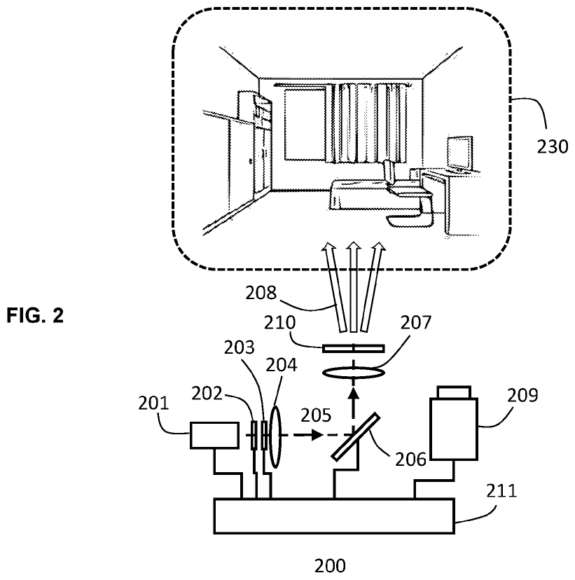

One configuration of a smart programmable ultraviolet germicidal irradiation system 200 in accordance with an example embodiment is shown in FIG. 2. Ultraviolet light 205 from a source 201 is collimated by optics 204 and is incident onto a beam steering device 206. A tunable attenuator 202 and a shutter 203 may be placed in the beam path. The shutter can act as an emergency beam block. The beam steering device is a UV-hardened device which can be, for example, a rotating drum, a rotating polygon, a spatial light modulator, a digital micromirror device or a mirror scanner which redirects the incoming light to output optics 207. The beam steering device 206 scans the beam over an area by modifying the direction of the light 208 to cover a range of a predefined area. One example of the predefined area is a bedroom 230. A camera 209 is also incorporated in the system 200. The source 201, tunable attenuator 202, shutter 203, beam steering device 206, and camera 209 are connected to a controller 211. An optical filter 210 can be used to filter out a part of the spectrum and prevent feedback to the source. In some embodiments, the camera is a simple RGB camera that can acquire color images or a UV camera that can measure UV illumination in real time. In other embodiments, the camera is a sophisticated imaging instrument that can incorporate an illuminating source, a hyperspectral camera, a SWIR camera, a MWIR camera, a LWIR camera, a spectrometer, an imaging polarimeter, and/or a 3D camera such as LIDAR system or plenoptic system. In yet other embodiments, the camera can be a network of multiple cameras located at different locations, and the scanning light sources can be a network of light sources at different locations.

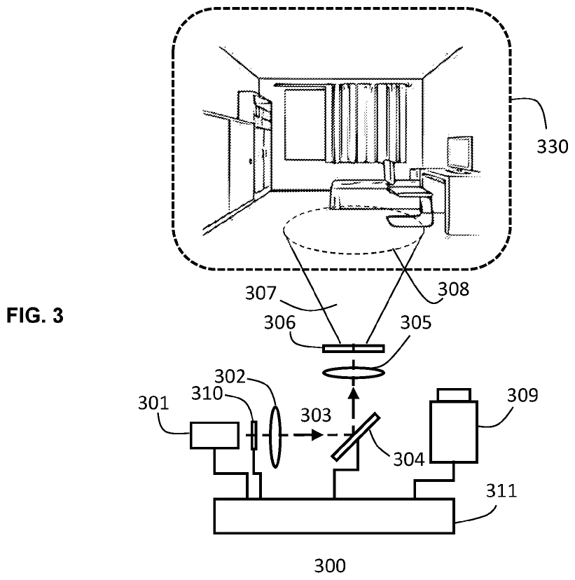

An alternate embodiment of a smart programmable ultraviolet germicidal irradiation system 300 is shown in FIG. 3. Ultraviolet light 303 from a source 301 is collimated by optics 302 and is incident onto a light modulation array 304. A shutter 310 may be placed in the beam path. The shutter can act as an emergency beam block. The light modulator array 304 is a UV-hardened device which can be, for example, a spatial light modulator or a digital micromirror device that projects a pattern of light to output optics 305. The light modulator array 304 directs a programmable patterned image of UV radiation 307 over an area 308 to cover different objects. One example of the area is a bedroom 330. A camera 309 is also incorporated in the system 300. The source 301, shutter 310, light modulator array 304, and camera 309 are connected to a controller 311. An optical filter 306 can be used to filter out a part of the spectrum and prevent feedback to the source. The system acts as a UV light projector with a functionality that is similar to that of a visible light projecting display.

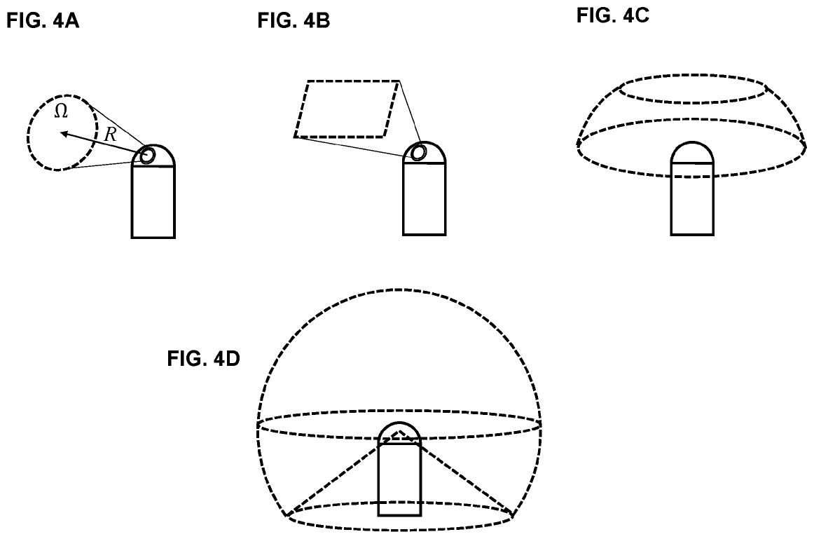

Both systems 200 and 300 can be designed with different projection optics and motion stages to cover a range of distances, R, and solid angles, Ω, depending on the application requirements. FIG. 4 shows different coverage arrangements for the UV sanitizer, which can range from a circular cone in FIG. 4A, to a rectangular cone in FIG. 4B, to a circular arc region in FIG. 4C and to a spherical region in FIG. 4D. These and other patterns can be produced in a variety of ways; for example, by selectively turning on or off the illumination beam (e.g., via turning on/off the DMD elements) that is projected onto the object of interest, by producing a particular pattern of illumination (e.g., a linear illumination pattern) that is scanned across a desired area and/or by introducing masks with different transmission patterns in the illumination path.

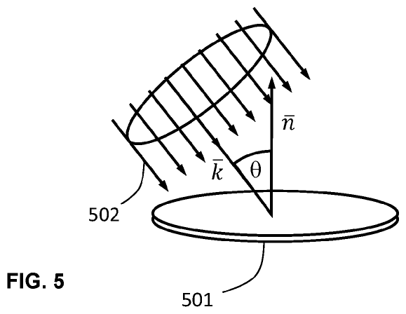

In addition to the illumination pattern, the disclosed embodiments enable a specific dosage to be provided to the target. The dosage incident on the target depends on the angle of incidence of the incoming UV light relative to the surface normal. FIG. 5 shows the surface of a target 501 with a normal direction defined by a vector n̅. Incident UV light 502 has a propagating direction k̅ making an angle θ relative to n̅, where

k̅·n̅=|k̅|n̅|cos (θ).

The incident light has the highest irradiance when k̅ is parallel to n̅ (the UV light is incident normal to the surface). The incident light has the lowest irradiance when k̅ is perpendicular to n̅ (the UV light is parallel to the surface). The irradiance E(θ) falling on the surface changes as the cosine of the incident angle θ, and is described by the Lambert's cosine law:

E(θ)=Eᵢcos(θ),

In the above equation, Eᵢ is the incident irradiance.

Exposure of UV light can irritate skin and eyes. In some embodiments, the system operates by actively sterilizing all areas inside the room, keeping track of the beam size and path and all motions insides the room and avoiding illumination of the occupant and objects that can be damaged by UV radiation. In some embodiments, the system can also keep track of locations of reflectors inside the room in order to avoid unwanted secondary illumination from the reflection. The location of reflectors can be provided to the system (e.g., if the locations are already known), can be determined via images of the area of interest, and/or can be monitored via live feeds and image recognition logic to identify and track the reflectors. For example, in the configurations of FIGS. 2 and 3, images taken by cameras 209 and 309 can be used to determine the location of the objects of interest (e.g., objects that should be sterilized and/or object that should not be irradiated). The premises can be further continuously or intermittently monitored to keep track of any changes in the location or orientation of those objects, or to determine if new objects are introduced into the premises. In some embodiments, the system can be installed in a vehicle, a plane, as well as in indoor and outdoor environments. In some embodiments, the system can be installed on a moving robot to disinfect hard-to-reach areas that are not in direct line-of-sight, on a fixed-position or fixed-rail system. For example, the system can be installed on an autonomous vacuum cleaner that moves around a room, on a different type of robotic or movable platform that can navigate around different parts of a room, or as part of a flying autonomous vehicle. In some embodiments, a compact version of the system can be incorporated in appliances such as a refrigerator, a washer or a dryer.

In some embodiments, the cameras 209 and 309 (shown in FIS. 2 and 3) can be used to conduct additional operations akin to calibration and determination of material properties of various objects. For example, all or parts of the room can be illuminated, and the reflected spectra can be measured by the camera(s) to determine the reflectivity of various objects, to determine whether or not the proper dosage is received by the objects of interest, and/or to determine whether the objects are fully or partially obstructed. In some embodiments, the cameras are used to monitor the premises during the exposure to detect, for example, any shifts in the objects' positioning or entry/presence of people or pets.

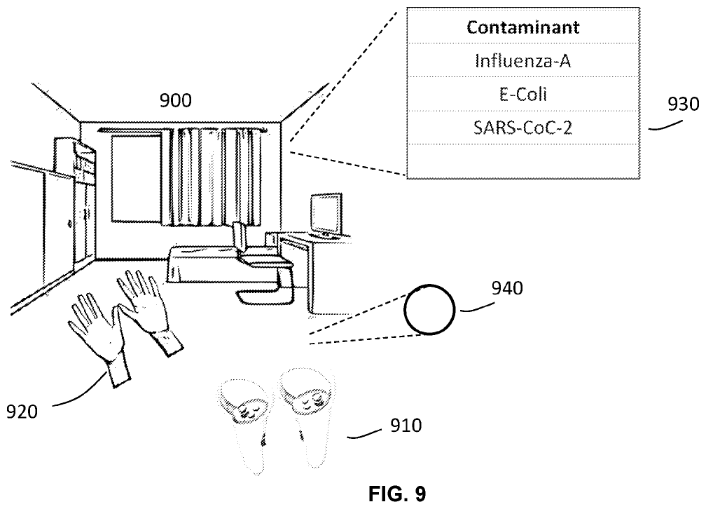

The disclosed systems and methods can be used to determine and administer the dosage delivered to an object for proper disinfection. The proper dosage can be determined based on the irradiance received by the target object (which depends on the angle between the illumination direction and the surface normal), the duration of the exposure, and the material properties (e.g., reflectivity) of the object's surface. For example, the angle of incidence can be obtained by either via image analysis, or via measuring the ratio of emitted and received irradiance for an object with known reflectivity. As another example, given the known position and orientation of an object, the reflectivity of the object can be determined by determining the reflected irradiance for a known incident irradiance. As noted earlier, different dosage values may be needed to irradiate different types of contaminants. Therefore, in some embodiments, the user can select the type of contaminant (e.g., virus or bacteria) that needs to be eliminated, and the system can determine, via computations and/or measurements, the dosage that is needed to sterilize the various objects of interest. For example, the user may select the type of contaminant via a user interface, such as a drop-down or pop-up menu (e.g., 930 in FIG. 9), may type or otherwise enter the name of contaminant, or conduct a search of a database of contaminants. Once the type or the name of the contaminant is specified, the system can obtain the required dosage (e.g., via retrieving values stored in a local or remote database and assuming a value of log kill), determine the required dosage, and irradiate the selected objects. It is expected that the output power of the UV source will change over the lifetime of the sanitizer, due to device degradation and environmental changes. The system can be designed to be self-calibrated in order to take into account the changes, such as source output power or beam direction. The determination of the exposure, including the exposure duration and irradiation dosage, will take into consideration the latest calibration.

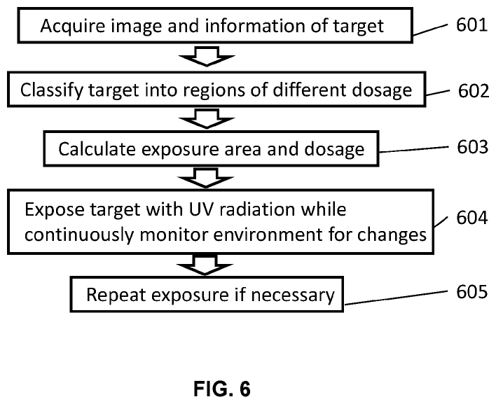

An example flow chart of the operations is shown in FIG. 6. The process of irradiation starts at 601 with the acquisition of image and information of the target area. This information can include but is not limited to location of objects or surfaces in the area, surface transmissions, reflection and absorption characteristics, refractive indices, Mueller matrices, and types of material of the objects or surfaces. At 602, regions in the area are classified based on dosage and type of pathogens or contaminants. For example, some regions may have zero dosage, while other regions may have a high dosage. At 603, the exposure area and the dosage are calculated based on the classification. In some embodiments, the classification and dosage can be input by the user or can be determined by algorithms, neural networks, and artificial intelligent systems. An exposure path, or the sequence in which different objects are exposed, can be calculated based on the different classified areas. The path can be set, for example, to minimize scan time or to maximize disinfection over certain areas. At 604, the target is exposed to the UV radiation for a predetermined duration of time to deliver the required dosage to the target. In some embodiments, as the exposure path of the light scanner is being executed, the target is continuously monitored for changes. In some embodiments, the UV exposure is stopped or is modified, if a person or pet is detected. At 605, the target area is re-exposed if needed.

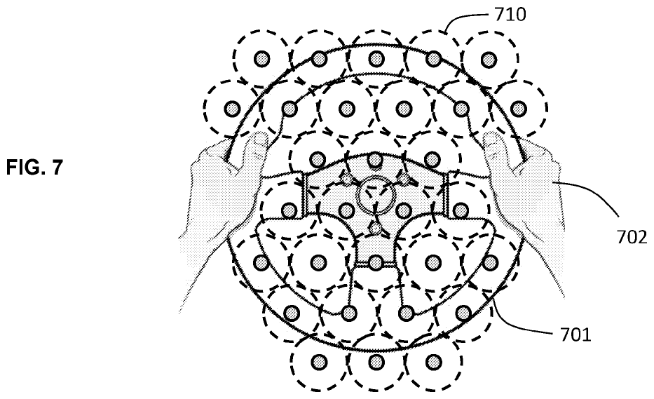

A disinfection example is illustrated in FIG. 7, where a steering wheel 701 is the target to be disinfected. A circular light beam 710 is scanned over the target area. Regions where the user's hands 702 are located are not exposed in this example. In other examples, the disinfection can be carried out when the user is not in the car or is not behind the wheel (e.g., overnight, and/or when the car is parked). The illumination source can be incorporated into a section of the roof; for example, as part of the dome light or in another location that allows illumination of the steering wheel from above.

The operation of the disinfection system requires initial calibration of the source, based on the location of the source and objects and the environment, with inputs (e.g., obtained from a user) such as the type of pathogens and start time. In some embodiments, the system can operate periodically in the evening or during a specific time window when no occupant is around. The system can have safety built-in features to prevent accidental UV exposures to the users/occupants and can remember the history of disinfecting different locations and targets. In some embodiments, the system may include temperature, humidity and motion sensors. For example, UV disinfection dosage can increase with relative humidity to compensate for water absorption. In some embodiments, the system can be voice activated or controlled by user from a remote location. For example, the user can ask the system to disinfect the surface of the red sofa in the room or the toilet seat and bathtub in a bathroom.

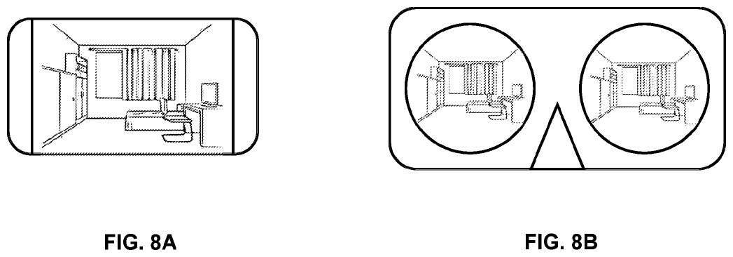

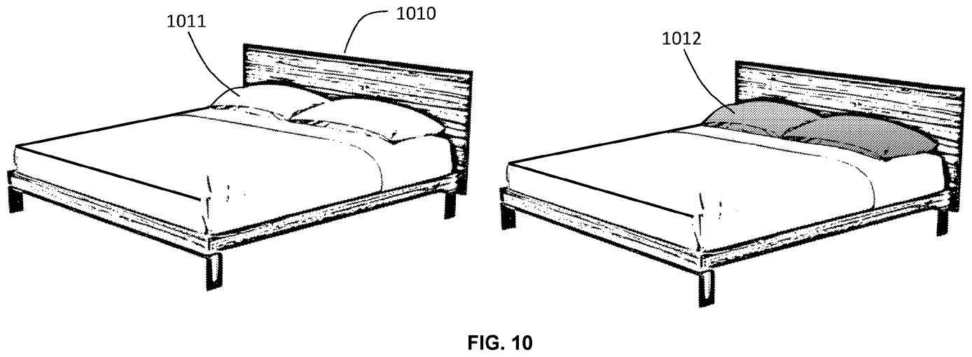

The system can also be controlled by using a virtual reality/augmented reality (AR/VR) interface such as a head mounted goggle, a portable computer, a tablet and a cell phone. For example, the user can mark different areas for disinfection using the interface. FIGS. 8A and 8B show a cell phone 800 and an AR/VR goggle device 810 which can be utilized to control the UV disinfection system (for example, while the user is in a different location away from the system to prevent accidental exposure). A user interface can be implemented on the portable device, such as on a cell phone 800. In some embodiments, the control of the operations of the UV disinfection system can be implemented on the user interface of an AR/VR goggle device 810 that can allow a user to operate the UV disinfection program using AR/VR controllers. FIG. 9 illustrates a set of AR/VR controllers that appear as a pair of hands 920 (selection icons) in the AR/VR world. The user can use the hands 920 to mark different surfaces in a room 900 for disinfection. Alternatively, the user can use the physical controllers 910 to direct a virtual marker beam inside the virtual room to mark different objects inside the virtual environment. For example, as shown in FIG. 10, the user can mark the pillows 1011 on a bed 1010. The user can then initiate the disinfection process by selecting a start/go button (and with an optional start time) to disinfect the surface of the marked pillows 1012 by the sanitization system. As noted earlier, the user may also select the type of contaminant to be disinfected by using, for example a pop-up menu 930. The selection can be carried out by the controllers 910, 920. In some embodiments, clicking or double-clicking on a menu entry may start the UV radiation. In other embodiment, the UV radiation may commence when a separate icon 940 is selected. In some embodiments, the AR/VR device can be a pair of glasses or other types of eyewear.

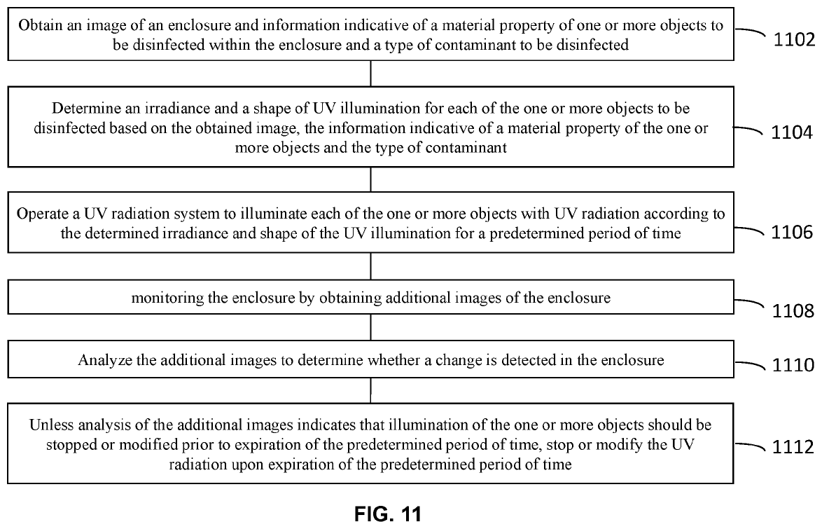

FIG. 11 illustrates a set of operations that can be carried out to disinfect one or more objects using ultraviolet radiation in accordance with an example embodiment. At 1102, an image of an enclosure and information indicative of a material property of the one or more objects to be disinfected within the enclosure and a type of contaminant to be disinfected are obtained. At 1104, an irradiance and a shape of UV illumination are determined for each of the one or more objects to be disinfected based on the obtained image, the information indicative of a material property of the one or more objects and the type of contaminant. At 1106, a UV radiation system is operated to illuminate each of the one or more objects with UV radiation according to the determined irradiance and shape of the UV illumination for a predetermined period of time. At 1108, the enclosure is monitored by obtaining additional images of the enclosure. At 1110, the additional images are analyzed to determine whether a change is detected in the enclosure. At 1112, unless analysis of the additional images indicates that illumination of the one or more objects should be stopped or modified prior to expiration of the predetermined period of time, the UV radiation is stopped or modified upon expiration of the predetermined period of time.

In one example embodiment, the information indicative of the material property of the one or more objects includes reflectivity of the one or more objects. In another example embodiment, determining the irradiance includes: obtaining a required dosage value based on the type of contaminant, obtaining a direction of a surface normal associated with each of the one or more objects, computing the irradiance based on an angle between the surface normal and direction of the UV illumination incident on each of the one or more objects, and determining an emitted irradiance by the UV illumination system to produce the required dosage at each of the one or more objects. In yet another example embodiment, the shape of the UV illumination for each of the one or more objects matches a surface area of the corresponding object. In still another example embodiment, the shape of the UV illumination includes one or more of a circular shape, rectangular shape, a hemispherical shape, a circular arc shape, or a spherical shape.

According to another example embodiment, the UV illumination system includes a movable scanning device that is used to produce the UV radiation according to the determined shape. In one example embodiment, the UV illumination system includes a spatial light modulator that is used to produce the UV radiation according to the determined irradiance and shape. In another example embodiment, analyzing the additional images results in detection of a change in the enclosure that indicates a presence of a person or an animal, and the above method further includes stopping or modifying the UV radiation. In yet another example embodiment, the above method further includes repeating the operating, monitoring, analyzing and stopping at a subsequent time.

In one example embodiment, determining the irradiance and the shape of UV illumination includes classification of the image into one or more regions requiring different exposures. In another example embodiment, the method further includes determining an exposure path based on the classification. In yet another example embodiment, the UV illumination comprises a pattern that includes regions with no UV radiation and regions with UV radiation having a particular irradiance.

Another aspect of the disclosed technology relates to a programmable ultraviolet disinfection system that includes a UV radiation source, a beam scanner or a spatial light modulator configured to produce an output beam having a particular shape, and a camera configured to capture images of an enclosure having one or more objects of interest, where the camera is further configured to detect UV radiation reflected from the one or more objects upon illumination by the UV radiation from the source. The system further includes a processor and a memory including instructions stored thereon, wherein the instructions upon execution by the processor cause the processor to obtain an image of an enclosure and information indicative of a material property of the one or more objects to be disinfected within the enclosure and a type of contaminant to be disinfected, and determine an irradiance and a shape of UV illumination for each of the one or more objects to be disinfected based on the obtained image, wherein the information is indicative of a material property of the one or more objects and the type of contaminant. The instructions upon execution by the processor further cause the processor to operate the programmable UV disinfection system to illuminate each of the one or more objects with UV radiation according to the determined irradiance and shape of the UV illumination for a predetermined period of time, monitor the enclosure by obtaining additional images of the enclosure, analyze the additional images to determine whether a change is detected in the enclosure, and unless analysis of the additional images indicates that illumination of the one or more objects should be stopped prior to expiration of the predetermined period of time, stop or modify the UV radiation upon expiration of the predetermined period of time.

In one example embodiment, the spatial light modulator is one of a digital micromirror device (DMD) or a liquid crystal modulator (LCM) and is configured to produce both the shape and the irradiance of the UV illumination for illuminating the one or more objects. In another example embodiment, the programmable UV disinfection system includes a tunable attenuator configured to selectively attenuate the UV illumination for illuminating the one or more objects.

In another example embodiment, the programmable UV disinfection system is incorporated as part of a movable platform configured to move along a path in the enclosure. In one example embodiment, the programmable UV disinfection system is incorporated as part of an illumination system in a car to disinfect a particular section of the car. In another example embodiment, the programmable UV disinfection system further includes a user interface on an electronic mobile device, wherein the user interface is configured to display a representation of the enclosure including the one or more objects of interest, enable a user to select the one or more objects of interest, commence the UV radiation to disinfect the one or more objects of interest.

According to an example embodiment, the user interface is a user interface of a mobile phone. In another example embodiment, the user interface is a user interface of an augmented reality (AR) or a virtual reality (VR) device. In still another example embodiment, the user interface is configured to select a contaminant that is to be eliminated.

Another aspect of the disclosed embodiments relates to a user interface configured to operate an ultraviolet (UV) disinfection system. The user interface is configured to display: a representation of an enclosure including the one or more objects of interest to be disinfected, and a selection icon configured to move across the representation of the enclosure and to allow selection or deselection of the one or more objects of interest, a menu comprising a listing of a plurality of contaminants or a text entry area that allows entry of a name of a contaminant, wherein the selection pointer allows a user to select one or more of the plurality of contaminants from the menu or to place a cursor in the text entry area. The user interface is further configured to display an activation mechanism configured to allow a user to commence the UV radiation to disinfect the one or more objects of interest or to stop the UV radiation.

In one example embodiment, the selection icon is a pair of hands. In another example embodiment, the menu and the selection pointer are controlled by voice command from user.

Various information and data processing operations described herein may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Therefore, the computer-readable media that is described in the present application comprises non-transitory storage media. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

The foregoing description of embodiments has been presented for purposes of illustration and description. The foregoing description is not intended to be exhaustive or to limit embodiments of the present invention to the precise form disclosed, and modifications and variations are possible in light of the above teachings or may be acquired from practice of various embodiments. The embodiments discussed herein were chosen and described in order to explain the principles and the nature of various embodiments and its practical application to enable one skilled in the art to utilize the present invention in various embodiments and with various modifications as are suited to the particular use contemplated. While operations are depicted in the drawings in a particular order, this should not be understood as requiring that such operations be performed in the particular order shown or in sequential order, or that all illustrated operations be performed, to achieve desirable results. The features of the embodiments described herein may be combined in all possible combinations of methods, apparatus, modules, and systems.

1. A method for disinfecting one or more objects using ultraviolet (UV) radiation, comprising:
obtaining an image of an enclosure and information indicative of a material property of the one or more objects to be disinfected within the enclosure and a type of contaminant to be disinfected;
determining an irradiance and a shape of UV illumination for each of the one or more objects to be disinfected based on the obtained image, the information indicative of a material property of the one or more objects and the type of contaminant;
operating a UV radiation system to illuminate each of the one or more objects with UV radiation according to the determined irradiance and shape of the UV illumination for a predetermined period of time;
monitoring the enclosure by obtaining additional images of the enclosure;
analyzing the additional images to determine whether a change is detected in the enclosure; and
unless analysis of the additional images indicates that illumination of the one or more objects should be stopped or modified prior to expiration of the predetermined period of time, stopping or modifying the UV radiation upon expiration of the predetermined period of time.

2. The method of claim 1, wherein the information indicative of the material property of the one or more objects includes reflectivity of the one or more objects.

3. The method of claim 1, wherein determining the irradiance includes:
obtaining a required dosage value based on the type of contaminant,
obtaining a direction of a surface normal associated with each of the one or more objects,
computing the irradiance based on an angle between the surface normal and direction of the UV illumination incident on each of the one or more objects, and
determining an emitted irradiance by the UV illumination system to produce the required dosage at each of the one or more objects.

4. The method of claim 1, wherein the shape of the UV illumination for each of the one or more objects matches a surface area of the corresponding object.

5. The method of claim 4, wherein the shape of the UV illumination includes one or more of a circular shape, rectangular shape, a hemispherical shape, a circular arc shape, or a spherical shape.

6. The method of claim 1, wherein the UV illumination system includes a movable scanning device that is used to produce the UV radiation according to the determined shape.

7. The method of claim 1, wherein the UV illumination system includes a spatial light modulator that is used to produce the UV radiation according to the determined irradiance and shape.

8. The method of claim 1, wherein analyzing the additional images results in detection of a change in the enclosure that indicates a presence of a person or an animal, and the method of claim 1 further includes stopping or modifying the UV radiation.

9. The method of claim 1, further including repeating the operating, monitoring, analyzing and stopping at a subsequent time.

10. The method of claim 1, wherein determining the irradiance and the shape of UV illumination includes classification of the image into one or more regions requiring different exposures.

11. The method of claim 10, further comprising determining an exposure path based on the classification.

12. The method of claim 1, wherein the UV illumination comprises a pattern that includes regions with no UV radiation and regions with UV radiation having a particular irradiance.

13. A programmable ultraviolet (UV) disinfection system, comprising:
a UV radiation source;
a beam scanner or a spatial light modulator configured to produce an output beam having a particular shape;
a camera configured to capture images of an enclosure having one or more objects of interest, the camera further configured to detect UV radiation reflected from the one or more objects upon illumination by the UV radiation from the source; and
a processor and a memory including instructions stored thereon, wherein the instructions upon execution by the processor cause the processor to:
obtain an image of an enclosure and information indicative of a material property of the one or more objects to be disinfected within the enclosure and a type of contaminant to be disinfected;
determine an irradiance and a shape of UV illumination for each of the one or more objects to be disinfected based on the obtained image, the information indicative of a material property of the one or more objects and the type of contaminant;
operate the programmable UV disinfection system to illuminate each of the one or more objects with UV radiation according to the determined irradiance and shape of the UV illumination for a predetermined period of time;
monitor the enclosure by obtaining additional images of the enclosure;
analyze the additional images to determine whether a change is detected in the enclosure; and
unless analysis of the additional images indicates that illumination of the one or more objects should be stopped prior to expiration of the predetermined period of time, stop or modify the UV radiation upon expiration of the predetermined period of time.

14. The programmable UV disinfection system of claim 13, wherein the spatial light modulator is one of a digital micromirror device (DMD) or a liquid crystal modulator (LCM) and is configured to produce both the shape and the irradiance of the UV illumination for illuminating the one or more objects.

15. The programmable UV disinfection system of claim 13, including a tunable attenuator configured to selectively attenuate the UV illumination for illuminating the one or more objects.

16. The programmable UV disinfection system of claim 13, wherein the programmable UV disinfection system is incorporated as part of a movable platform configured to move along a path in the enclosure.

17. The programmable UV disinfection system of claim 13, wherein the programmable UV disinfection system is incorporated as part of an illumination system in a car to disinfect a particular section of the car.

18. The programmable UV disinfection system of claim 13, further including a user interface on an electronic mobile device, wherein the user interface is configured to:
display a representation of the enclosure including the one or more objects of interest;
enable a user to select the one or more objects of interest; and
commence the UV radiation to disinfect the one or more objects of interest.

19. The programmable UV disinfection system of claim 18, wherein the user interface is a user interface of a mobile phone.

20. The programmable UV disinfection system of claim 18, wherein the user interface is a user interface of an augmented reality (AR) or a virtual reality (VR) device.

21. The programmable UV disinfection system of claim 18, wherein the user interface is configured to select a contaminant that is to be eliminated.

22. A user interface configured to operate an ultraviolet (UV) disinfection system, the user interface configured to display:
a representation of an enclosure including the one or more objects of interest to be disinfected;
a selection icon configured to move across the representation of the enclosure and to allow selection or deselection of the one or more objects of interest;
a menu comprising a listing of a plurality of contaminants or a text entry area that allows entry of a name of a contaminant, wherein the selection pointer allows a user to select one or more of the plurality of contaminants from the menu or to place a cursor in the text entry area; and
an activation mechanism configured to allow a user to commence the UV radiation to disinfect the one or more objects of interest or to stop the UV radiation.

23. The user interface of claim 22, wherein the selection icon is a pair of hands.

24. The user interface of claim 22, wherein the menu and the selection pointer are controlled by voice command from user.
